(19) United States
(12) Patent Application Publication
(10) Pub. No.: US 2019/0000307 A1
(43) Pub. Date: Jan. 3, 2019

(54) OPTICAL TRANSMITTER AND ENDOSCOPE

(71) Applicant: OLYMPUS CORPORATION, Tokyo (JP)

(72) Inventors: Kosuke KAWAHARA, Kamiina-gun (JP); Yusuke NAKAGAWA, Kamiina-gun (JP)

(73) Assignee: OLYMPUS CORPORATION, Tokyo (JP)

(21) Appl. No.: 16/127,794

(22) Filed: Sep. 11, 2018

Related U.S. Application Data
(63) Continuation of application No. PCT/JP2016/058143, filed on Mar. 15, 2016.

(51) Int. Cl. A61B 1/04; G02B 23/24; A61B 1/00

(52) U.S. Cl. CPC A61B 1/04; G02B 23/2407; A61B 1/00096; A61B 1/00126; A61B 1/00009

(57) ABSTRACT

An optical transmitter includes an optical device, a wiring board in which the optical device and an electronic component are mounted on a first primary surface, an optical waveguide plate in which an upper surface is bonded to the wiring board and an optical waveguide is photo-coupled with the optical device by a reflection portion, an optical fiber photo-coupled with the optical waveguide, and a conductor, a first groove and a second groove each having an opening in one of side surfaces are provided on the upper surface of the optical waveguide plate, the optical fiber is inserted into the first groove, and the conductor is inserted into the second groove and is bonded to an electrode on a second primary surface of the wiring board immediately above the second groove.

### CROSS REFERENCE TO RELATED APPLICATION

This application is a continuation application of PCT/JP2016/058143 filed on May 15, 2016, the entire contents of which are incorporated herein by this reference.

### BACKGROUND OF INVENTION

### Field of the Invention

The present invention relates to an optical transmitter including an optical device, a wiring board on which the optical device and an electronic component are mounted, an optical waveguide plate bonded to the wiring board, an optical fiber photo-coupled with the optical device through the optical waveguide plate, and a conductor connected to the wiring board, and to an endoscope having the optical transmitter.

The endoscope has an image pickup device such as a CCD on a distal end rigid portion of an elongated insertion portion. Recently, use of the image pickup device having a high pixel number in the endoscope has been examined When an image pickup device with a high pixel number is used, a signal amount to be transmitted from the image pickup device to a signal processing device (processor) is increased and thus, optical signal transmission through a fine optical fiber by an optical signal using the optical transmitter is preferable to electric signal transmission through metal wiring by an electric signal.

The optical transmitter has an optical device, a wiring board on which the optical device is surface-mounted on a first primary surface, an optical waveguide plate made to adhere to a second primary surface of the wiring board, and an optical fiber. The optical device generates an optical signal by a driving signal from a signal cable bonded to the wiring board, for example. The optical signal is guided to the optical fiber through the optical waveguide. That is, from a rear end surface of the optical transmitter, the optical fiber for guiding the optical signal and the signal cable and the optical fiber for transmitting the electric signal are extended.

For lower invasiveness of an endoscope, size reduction (reduction in diameter/reduction in length) of the optical transmitter is in demand.

Japanese Patent Application Laid-Open Publication No. 2009-222749 discloses an optical transmitter including a photoelectric composite cable which integrates the optical fiber and the signal cable. In the photoelectric composite cable, a metal coated layer disposed on an outer peripheral part of the optical fiber transmits the electric signal.

### SUMMARY OF THE INVENTION

An optical transmitter of an embodiment of the present invention is an optical transmitter including an optical device having a light emitting portion configured to output light of an optical signal or a light receiving portion into which the light of the optical signal is inputted, a wiring board having a first primary surface and a second primary surface facing the first primary surface and in which the optical device and an electronic component are mounted on the first primary surface, an optical waveguide plate having an upper surface and a lower surface facing the upper surface, in which the upper surface is bonded to the second primary surface of the wiring board, and the optical waveguide formed in a direction parallel with the upper surface is photo-coupled with the optical device by a reflection portion, a substrate having a front surface and a rear surface facing the front surface, in which the lower surface of the optical waveguide plate is disposed on the front surface, an optical fiber, a distal end surface of which is arranged facing an end surface of the optical waveguide of the optical waveguide plate, the optical fiber being photo-coupled with the optical waveguide, and conductors bonded to the wiring board, in which a first groove and second grooves having openings on one of side surfaces are provided on the upper surface of the optical waveguide plate, the openings of the first groove and the second grooves are covered by the second primary surface of the wiring board, a distal end portion of the optical fiber is inserted into a first hole formed by the second primary surface of the wiring board and a wall surface of the first groove, and the conductors are inserted into second holes formed by the second primary surface of the wiring board and wall surfaces of the second grooves and bonded to electrodes on the second primary surface of the wiring board immediately above the second grooves.

An endoscope of another embodiment of the present invention includes an optical transmitter. The optical transmitter includes an optical device having a light emitting portion configured to output light of an optical signal or a light receiving portion into which the light of the optical signal is inputted, a wiring board having a first primary surface and a second primary surface facing the first primary surface and in which the optical device and an electronic component are mounted on the first primary surface, an optical waveguide plate having an upper surface and a lower surface facing the upper surface, in which the upper surface is bonded to the second primary surface of the wiring board, and the optical waveguide formed in a direction parallel with the upper surface is photo-coupled with the optical device by a reflection portion, a substrate having a front surface and a rear surface facing the front surface, in which the lower surface of the optical waveguide plate is disposed on the front surface, an optical fiber, a distal end surface of which is arranged facing an end surface of the optical waveguide of the optical waveguide plate, the optical fiber being photo-coupled with the optical waveguide, and conductors bonded to the wiring board, in which a first groove and second grooves having openings on one of side surfaces are provided on the upper surface of the optical waveguide plate, the openings of the first groove and the second grooves are covered by the second primary surface of the wiring board, a distal end portion of the optical fiber is inserted into a first hole formed by the second primary surface of the wiring board and a wall surface of the first groove, and the conductors are inserted into second holes formed by the second primary surface of the wiring board and wall surfaces of the second grooves and bonded to electrodes on the second primary surface of the wiring board immediately above the second grooves.

### BRIEF DESCRIPTION OF THE DRAWINGS

FIG. 1 is an exploded perspective view of an optical transmitter of a first embodiment;

FIG. 2 is a sectional view along a II-II line in FIG. 1 of the optical transmitter of the first embodiment;

FIG. 3 is a sectional view along a line in FIG. 1 of the optical transmitter of the first embodiment;

FIG. 4 is a sectional view along a IV-IV line in FIG. 1 of the optical transmitter of the first embodiment;

FIG. 5 is a sectional view of the optical transmitter of a modification of the first embodiment;

FIG. 6 is a sectional view of an optical transmitter of a second embodiment;

FIG. 7 is a sectional view of an optical transmitter of a modification 1 of the second embodiment;

FIG. 8 is a sectional view of an optical transmitter of a modification 2 of the second embodiment;

FIG. 9 is a sectional view of an optical transmitter of a modification 3 of the second embodiment;

FIG. 10 is a sectional view of an optical transmitter of a modification 4 of the second embodiment; and

FIG. 11 is an appearance view of an endoscope of a third embodiment.

### DETAILED DESCRIPTION OF THE PREFERRED EMBODIMENTS

### First Embodiment

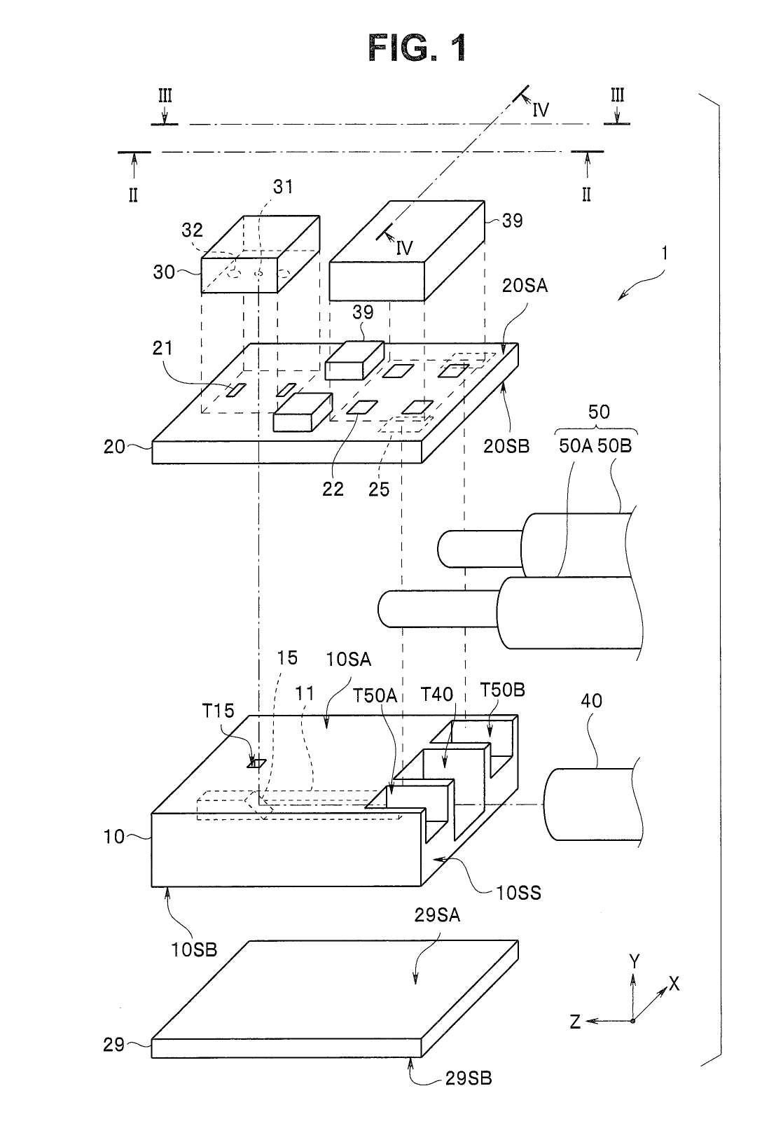

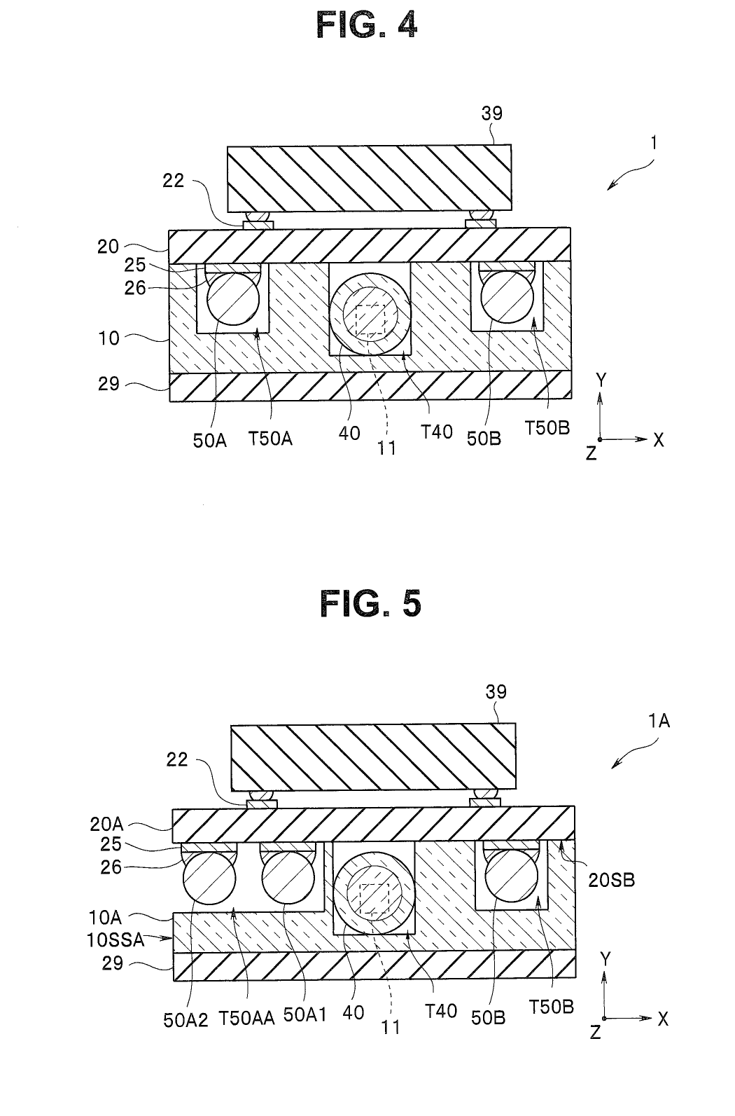

An optical transmitter 1 of a first embodiment will be described by using FIG. 1 to FIG. 4. In the following description, note that figures based on each of the embodiments are schematic, and relationships between thicknesses and widths of each portion, ratios of the thicknesses among respective portions and the like are different from actual ones, and the relationships of dimensions and the ratios of portions are different from each other among the figures in some cases. Illustration of some constituent elements is omitted in some cases. Note that a direction of an optical device with respect to an optical waveguide plate, that is, a direction where a value of a Y-axis increases is referred to as an “upper” direction (see FIG. 1 and the like).

The optical transmitter 1 includes an optical device 30, a wiring board 20, an optical waveguide plate 10, a substrate 29, an optical fiber 40, and conductors 50A and 50B.

In the following, when each of a plurality of constituent elements having the same function is referred to, one alphabet letter at the end of reference numeral is omitted in some cases. For example, each of the conductors 50A and 50B is referred to as a conductor 50.

In the optical transmitter 1, the optical device 30 is a light emitting element. That is, the optical device 30 is a VCSEL (vertical cavity surface emitting LASER) having a light emitting portion 31 outputting light of an optical signal to a light emitting surface 30SA, for example. For example, the super small-sized optical device 30 having a dimension on a plan view of 250 μm×300 μm has the light emitting portion 31 having a diameter of 20 μm and two external electrodes 32 for supplying a driving signal to the light emitting portion 31 on the light emitting surface 30SA. The optical device 30 projects light to the light emitting surface 30SA in a perpendicular direction (Y-axis direction).

The wiring board 20 and the substrate 29 are flexible FPCs (flexible printed circuits) wiring board having a resin such as polyimide as a base. The substrate 29 having a front surface 29SA and a rear surface 29SB facing the front surface 29SA are a base of the optical waveguide plate 10.

The wiring board 20 has a first primary surface 20SA and a second primary surface 20SB facing the first primary surface 20SA, and the external electrode 32 of the optical device 30 is ultrasound-bonded to a bond electrode 21 of the first primary surface 20SA. On an electrode 22 of the first primary surface 20SA, an electronic component 39 such as a chip capacitor and driver IC is also mounted. Note that as will be described later, the wiring board 20 is a double-faced wiring board also having a plurality of electrodes 25 on the second primary surface 20SB.

The optical waveguide plate 10 has an upper surface 10SA and a lower surface 10SB facing the upper surface 10SA. The polymer type optical waveguide plate 10 has a core 11 made of a first resin having a refractive index of n1 and a clad 12 made of a second resin having a refractive index of n2 surrounding a periphery of the core 11 as main constituent members. And n1>n2. For efficient optical transmission, a difference between the refractive index n1 of the core 11 and the refractive index n2 of the clad 12 is preferably 0.05 or more and 0.20 or less. The core 11 constitutes an optical waveguide which is an optical path for guiding an optical signal. The core (optical waveguide) 11 is formed in a direction parallel with the upper surface 10SA.

The core 11 and the clad 12 are made of fluorinated polyimide resin excellent in heat resistance, transparency and isotropy and having a refractive index of 1.60 to 1.75, for example.

The polymer type optical waveguide plate is easier to be machined and more flexible than an optical waveguide plate made of an inorganic material such as quartz. Thus, the optical waveguide plate of the optical transmitter 1 is preferably a polymer type.

On the upper surface 10SA of the optical waveguide plate 10, a first groove T40 and second grooves T50 (T50A, T50B) having openings in a side surface 10SS are provided. The first groove T40 and the second grooves T50 are formed in the clad 12 and do not reach the core 11. The first groove T40 and the second grooves T50 are U-grooves each having a substantially square section but may be V-grooves each having a triangular section.

The second primary surface 20SB of the wiring board 20 is bonded to the upper surface 10SA of the optical waveguide plate 10 through a resin adhesive layer (not shown) so as to close the first groove T40 and the second grooves T50. Thus, the first groove T40 is a first hole having the opening in the side surface 10SS. The second grooves T50 are second holes having openings in the side surface 10SS.

The substrate 29 is disposed on the lower surface 10SB of the optical waveguide plate 10. The substrate 29 is a support substrate when the optical waveguide plate is fabricated and may be peeled off the optical waveguide plate 10 after the fabrication. That is, the substrate 29 is not an indispensable constituent element of the optical transmitter 1.

The optical fiber 40 is a multi-mode fiber having a core diameter of 50 μm and a clad diameter of 125 μm. The optical fiber 40 is inserted into the first groove T40 of the optical waveguide plate 10 and is fixed by an ultraviolet curable resin (not shown), for example. That is, the optical fiber 40 has a distal end surface arranged by facing an end surface of the optical waveguide 11 and is photo-coupled with the optical waveguide 11. A section of the optical waveguide 11, that is, a size of the core 11 is preferably equal to or slightly smaller than the core diameter of the optical fiber 40. When the diameter of a core 41 of the optical fiber 40 is 50 μm, for example, a sectional shape of the core 11 is a regular square with each side of 45 μm.

A prism 15 which is a reflection portion is disposed in a recess portion T15 of the core (optical waveguide) 11 immediately below the optical device 30 (light emitting portion 31). The prism 15 reflects an optical signal of an optical path O1 projected by the optical device 30 in a direction (Y-direction) orthogonal to the upper surface 10SA of the optical waveguide plate 10, that is, in a direction orthogonal to an extended direction of the core (optical waveguide) 11 and guides the light to an optical path O2 in an extended direction (Z-direction) of the core (optical waveguide) 11. The optical signal is incident to the optical fiber 40 through the core 11 which is an optical waveguide and is guided.

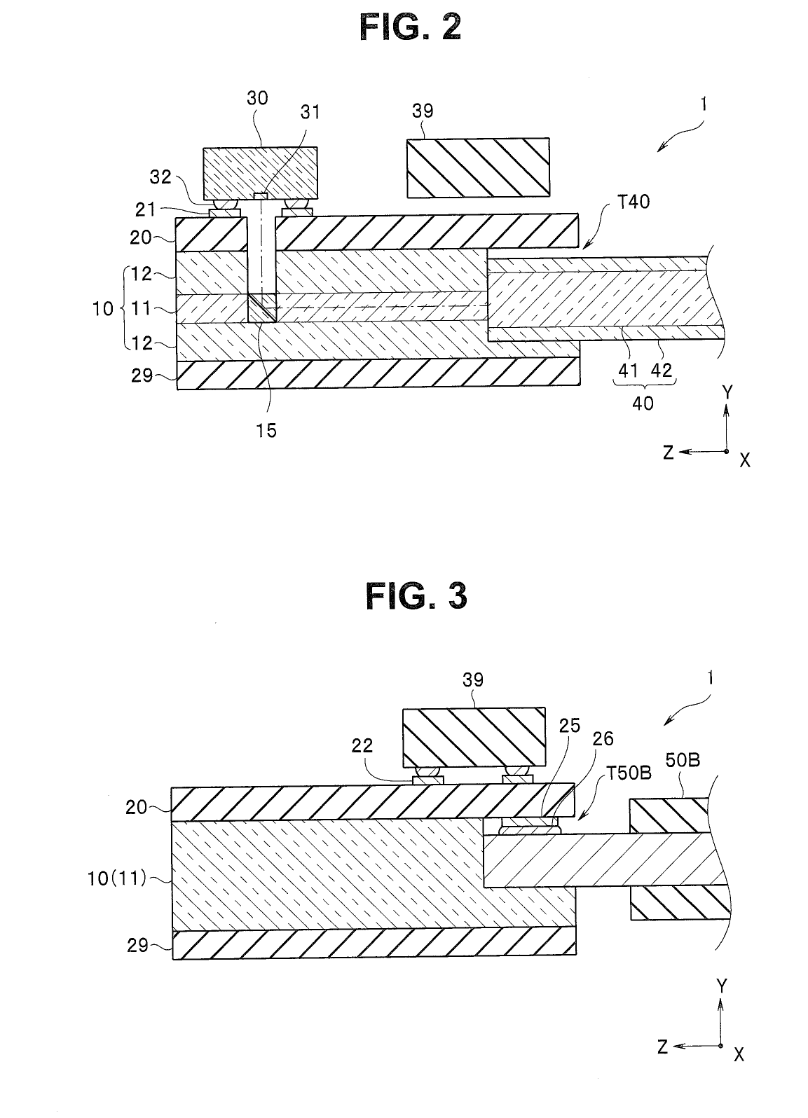

As illustrated in FIG. 2 and the like, a portion which becomes the optical path O1 of the wiring board 20 and the optical waveguide plate 10 is hollow like the recess portion T15. However, a transparent resin may be filled in the recess portion T15 or a through hole does not have to be formed in the wiring board 20 having high transmittance.

The reflection portion may be a wall surface of the V-groove formed in the second primary surface 20SB of the optical waveguide plate 10, for example, as long as the optical path O1 and the optical path O2 can be photo-coupled.

On the second primary surface 20SB of the wiring board 20, the electrode 25 electrically connected to the optical device 30 is disposed. That is, the electrode 25 of the second primary surface 20SB is electrically connected to the electrode 22 and the bond electrode 21 of the first primary surface 20SA through a through wire, not shown.

As already described, the first groove T40 and the second grooves T50 of the optical waveguide plate 10 are holes in which the upper surfaces are closed by the wiring board 20. An internal dimension of the first hole of the first groove T40 is slightly larger than an outer diameter of the optical fiber. The internal dimension of the second hole of the second groove T50 is slightly larger than an outer diameter of the conductor 50. Thus, when the optical fiber 40 is inserted into the first hole, an outer peripheral surface of the optical fiber 40 is brought into contact with the wall surface of the groove and thus, positioning is carried out automatically.

The conductor 50 inserted into the second hole is bonded with a solder 26, for example, to the electrode 25 on the second primary surface of the wiring board 20 which is the upper surface of the hole. Since the conductor 50 is temporarily held by being inserted into the second hole, bonding is easy.

Since the upper surfaces of the first groove T40 and the second grooves T50 of the optical waveguide plate 10 are closed by the wiring board 20, the electronic component 39 is also mounted on the first primary surface 20SA immediately above the grooves. That is, the first primary surface 20SA of an area immediately above the grooves of the wiring board 20 is an electronic component mounting area, and the second primary surface 20SB is an area where the conductor 50 is bonded.

The optical transmitter 1 in which the conductors 50 are inserted into the second grooves T50 formed in the clad 12 of the optical waveguide plate 10 has a diameter smaller than an optical transmitter in which the conductors 50 are bonded to the first primary surface of the wiring board. Moreover, the optical transmitter 1 is small and short since the electronic component 39 is mounted on the whole surface of the first primary surface 20SA of the wiring board 20.

In the description above, a case where the optical device 30 is a light emitting element, that is, the E/O optical transmitter 1 which converts the electric signal to the optical signal was described. However, even if the optical device is a light receiving element such as a PD having a light receiving portion into which the light of the optical signal is inputted, that is, even if the optical device is an O/E optical transmitter which converts the optical signal to the electric signal, it has the same effect as long as the O/E optical transmitter has the same configuration (the conductor is inserted in the second groove and is bonded to the electrode on the second primary surface of the wiring board immediately above the second groove) as the optical transmitter 1.

Needless to say, the same effect is exerted even in the case of the optical transmitter having the light emitting element and the light receiving element or the optical transmitter having a plurality of the light emitting elements or a plurality of the light receiving elements as long as the optical transmitter has the same configuration as the optical transmitter 1.

### Modification of First Embodiment

An optical transmitter 1A of a modification of the first embodiment is similar to the optical transmitter 1 and has the same effect and thus, the same reference numerals are given to the same constituent elements, and description will be omitted.

As illustrated in FIG. 5, two second grooves T50AA and T50B are provided on an optical waveguide plate 10A of the optical transmitter 1A. The second groove T50AA is a notch also having an opening in a side surface 10SSA orthogonal to a side surface 10SS. And two conductors 50A1 and 50A2 are inserted into the second groove T50AA. The conductors 50A1 and 50A2 are bonded to the electrode 25 of the second primary surface 20SB of a wiring board 20A, respectively, with the solder 26. The conductor 50B is inserted into the second groove T50B and is bonded to the electrode 25.

Two of the three conductors 50A1, 50A2, and 50B are driving signal supply lines of the optical device 30 and one is an earth potential line, for example.

That is, the wiring board 20 of the optical transmitter may be bonded to two or more conductors 50. A plurality of the conductors 50 may be inserted into one groove of the optical waveguide plate.

The optical transmitters 1 and 1A have the two second grooves T50A (T50AA) and T50B with the first groove T40 being interposed in between, but the optical transmitter may have the second groove T50AA into which the two conductors 50 are inserted on one of side surface sides of the first groove T40 and does not have to have the second groove T50B on the other side surface side.

### Second Embodiment

An optical transmitter 1B of a second embodiment is similar to the optical transmitter 1 and has the same effect and thus, the same reference numerals are given to the same constituent elements, and description will be omitted.

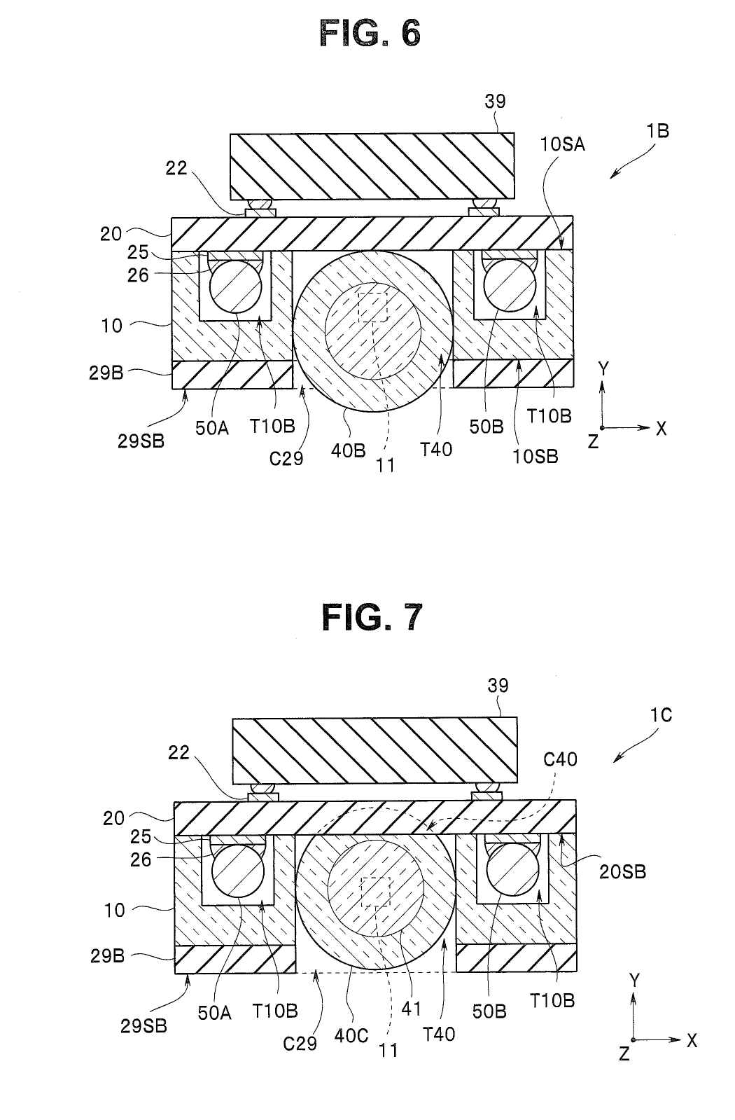

As illustrated in FIG. 6, an optical fiber 40B of the optical transmitter 1B is thicker than the optical fiber 40 and an outer peripheral surface protrudes from a rear surface 29SB of a substrate 29B through a notch C29 of the substrate 29B.

The groove T40 into which the optical fiber 40B of the optical transmitter 1B is inserted is a through hole penetrating the first primary surface 10SA and the second primary surface 10SB in the optical waveguide plate 10. That is, the groove T40 may be a through groove.

### Modifications of Second Embodiment

Optical transmitters 1C to 1F in modifications 1 to 4 of the second embodiment are similar to the optical transmitter 1B and have the same effect and thus, the same reference numerals are given to the same constituent elements, and the description will be omitted.

### Modification 1 of Second Embodiment

As illustrated in FIG. 7, an optical fiber 40C of the optical transmitter 1C is thicker than the optical fiber 40 but has a notch C40 chamfered in a direction parallel with a long axis on an outer peripheral surface of a distal end portion. The notch C40 is formed so as not to give a bad influence on the core 41 of the optical fiber 40C.

Since the outer peripheral surface of the optical fiber 40C does not protrude from the rear surface 29SB of the substrate 29B, the optical transmitter 1C is smaller in height (Y-direction dimension) than the optical transmitter 1B. Moreover, since the optical fiber 40 is arranged so that the center of the optical fiber 40 substantially matches a center of the core (optical waveguide) 11 of the optical waveguide plate 10, bonding efficiency can be improved.

In the optical transmitter 1C, since a notched surface of the optical fiber 40C is brought into contact with the second primary surface 20SB of the second primary surface, positioning of the optical fiber 40C in a rotation direction can be carried out easily.

### Modification 2 of Second Embodiment

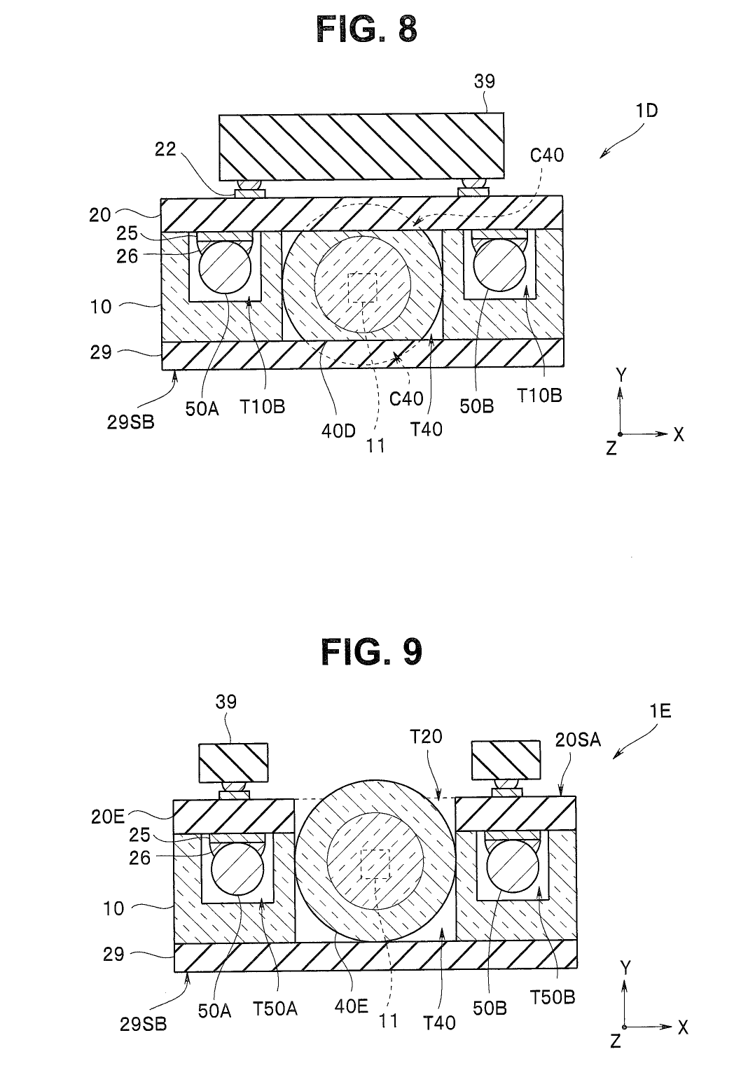

As illustrated in FIG. 8, an optical fiber 40D of an optical transmitter 1D is thicker than the optical fiber 40 but has the chamfered portions C40 chamfered in a direction parallel with a long axis on both facing side surfaces of a distal end portion.

The optical fiber 40D is accommodated in the first groove T40 without providing a notch in the wiring board 20 or the substrate 29.

Since the notched surface of the optical fiber 40D is brought into contact with the wiring board 20 and the substrate 29, positioning of the rotation direction can be carried out easily.

### Modification 3 of Second Embodiment

As illustrated in FIG. 9, an optical fiber 40E of an optical transmitter 1E is thicker than the optical fiber 40 and a side surface protrudes from the first primary surface 20SA of a wiring board 20E through a notch T20 of the wiring board 20E.

The protruding side surface of the optical fiber 40E is lower than an upper surface of the electronic component 39 mounted on the first primary surface 20SA. Thus, the protrusion of the optical fiber 40E does not have an influence on a height of the optical transmitter 1E.

Since a side surface of the optical fiber 40E protrudes from the wiring board 20E through the notch T20, the electronic component cannot be mounted immediately above the first groove T40 of the wiring board 20E. However, the electronic component 39 is mounted on the second groove T50.

If the electronic component 39 is mounted on the first primary surface 10SA immediately above at least either one of the first groove or the second groove of the wiring board, the optical transmitter can be made shorter and smaller.

### Modification 4 of Second Embodiment

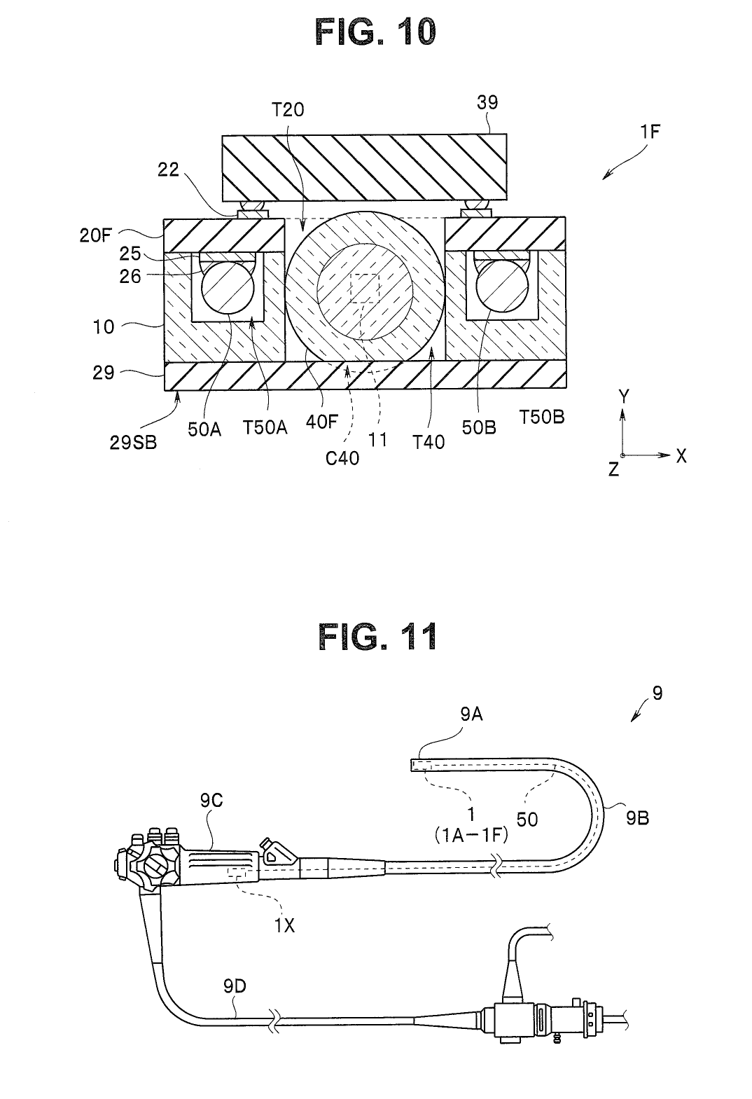

As illustrated in FIG. 10, an optical fiber 40F of an optical transmitter 1F is thicker than the optical fiber 40, but an outer peripheral surface has the notch C40 chamfered in the direction parallel with a long axis. Thus, the outer peripheral surface of the optical fiber 40F protrudes only slightly from the first primary surface 20SA of the wiring board 20F.

The electronic component 39 is mounted on the wiring board 20F so as to cross the notch T20.

In the optical transmitter 1F, since the notched surface of the optical fiber 40F is brought into contact with the substrate 29, positioning of the optical fiber 40F in the rotation direction can be also carried out. Needless to say, the notch C40 may be provided on an upper side and a lower side of the optical fiber 40F.

Needless to say, the optical transmitters 1B to 1F also have the same effect as long as the optical transmitters 1B to 1F have the configuration of the optical transmitter 1A and the like in the modification of the first embodiment.

### Third Embodiment

An endoscope 9 of a third embodiment will be described. Since the optical transmitters 1 and 1A to 1F of the endoscope 9 are the same as the optical transmitter 1 and the like of the embodiments, description will be omitted. The endoscope 9 having the optical transmitter 1 will be described below as an example.

As illustrated in FIG. 11, the endoscope 9 includes an insertion portion 9B in which an image pickup portion having an image pickup device with a high pixel number is disposed on a distal end rigid portion 9A, an operation portion 9C disposed on a base end side of the insertion portion 9B, and a universal cord 9D extending from the operation portion 9C.

An electric signal outputted by the image pickup device is converted to an optical signal by the optical transmitter 1 in which the optical device is a planar light emitting laser, the optical device disposed on the operation portion 9C through the optical fiber 40 is converted to the electric signal again by an optical transmitter 1X which is a PD and is transmitted through metal wiring (not shown). That is, the signal is transmitted through the optical fiber 40 in the insertion portion 9B having a small diameter.

The optical transmitter 1 is super small-sized and can be manufactured easily. Thus, the endoscope 9 has the distal end portion 9A and the insertion portion 9B having small diameters but can be manufactured easily.

The optical transmitter 1X has a relatively wide arrangement space but preferably has the same configuration as the optical transmitter 1.

The present invention is not limited to the aforementioned embodiments and modifications but is capable of various changes, combinations and applications within a range not departing from a gist of the invention.

What is claimed is:

1. An optical transmitter comprising:
an optical device having a light emitting portion configured to output light of an optical signal or a light receiving portion into which the light of the optical signal is inputted;
a wiring board having a first primary surface and a second primary surface facing the first primary surface and in which the optical device and an electronic component are mounted on the first primary surface;
an optical waveguide plate having an upper surface and a lower surface facing the upper surface, in which the upper surface is bonded to the second primary surface of the wiring board, and the optical waveguide formed in a direction parallel with the upper surface is photo-coupled with the optical device by a reflection portion;
a substrate having a front surface and a rear surface facing the front surface, in which the lower surface of the optical waveguide plate is disposed on the front surface;
an optical fiber, a distal end surface of which is arranged facing an end surface of the optical waveguide of the optical waveguide plate, the optical fiber being photo-coupled with the optical waveguide; and
conductors bonded to the wiring board, wherein
a first groove and second grooves having openings on one of side surfaces are provided on the upper surface of the optical waveguide plate;
the openings of the first groove and the second grooves are covered by the second primary surface of the wiring board;
a distal end portion of the optical fiber is inserted into a first hole formed by the second primary surface of the wiring board and a wall surface of the first groove; and
the conductors are inserted into second holes formed by the second primary surface of the wiring board and wall surfaces of the second grooves and bonded to electrodes on the second primary surface of the wiring board immediately above the second grooves.

2. The optical transmitter according to claim 1, wherein
the electronic component is mounted on the first primary surface of the wiring board immediately above at least one of the first groove or the second grooves.

3. The optical transmitter according to claim 2, wherein
the second grooves are provided with the first groove being interposed in between.

4. The optical transmitter according to claim 3, wherein
an outer peripheral surface of the optical fiber protrudes from the rear surface of the substrate through a notch in the substrate.

5. The optical transmitter according to claim 3, wherein
an outer peripheral surface of the optical fiber is chamfered in a direction parallel with a long axis; and
the outer peripheral surface of the optical fiber does not protrude from the rear surface of the substrate.

6. The optical transmitter according to claim 3, wherein
an outer peripheral surface of the optical fiber protrudes from the first primary surface of the wiring board through a notch in the wiring board.

7. The optical transmitter according to claim 6, wherein
the outer peripheral surface of the optical fiber is chamfered in a direction parallel with a long axis.

8. An endoscope comprising an optical transmitter,
the optical transmitter including:
an optical device having a light emitting portion configured to output light of an optical signal or a light receiving portion into which the light of the optical signal is inputted;
a wiring board having a first primary surface and a second primary surface facing the first primary surface and in which the optical device and an electronic component are mounted on the first primary surface;
an optical waveguide plate having an upper surface and a lower surface facing the upper surface, in which the upper surface is bonded to the second primary surface of the wiring board, and the optical waveguide formed in a direction parallel with the upper surface is photo-coupled with the optical device by a reflection portion;
a substrate having a front surface and a rear surface facing the front surface, in which the lower surface of the optical waveguide plate is disposed on the front surface;
an optical fiber, a distal end surface of which is arranged facing an end surface of the optical waveguide of the optical waveguide plate, the optical fiber being photo-coupled with the optical waveguide; and
conductors bonded to the wiring board, wherein
a first groove and second grooves having openings on one of side surfaces are provided on the upper surface of the optical waveguide plate;
the openings of the first groove and the second grooves are covered by the second primary surface of the wiring board;
a distal end portion of the optical fiber is inserted into a first hole formed by the second primary surface of the wiring board and a wall surface of the first groove; and
the conductors are inserted into second holes formed by the second primary surface of the wiring board and wall surfaces of the second grooves and bonded to electrodes on the second primary surface of the wiring board immediately above the second grooves.
